(19) United States
(12) Patent Application Publication
(10) Pub. No.: US 2025/0090783 A1
(43) Pub. Date: Mar. 20, 2025

(54) Electronic Nasal Cannula Flame Detector And Oxygen Shutoff Device

(71) Applicant: United States Government As Represented By The Department Of Veterans Affairs, Washington, DC (US)

(72) Inventors: David Beck, Tampa, FL (US); Mark Beck, Tampa, FL (US)

(21) Appl. No.: 18/886,302

(22) Filed: Sep. 16, 2024

Related U.S. Application Data
(60) Provisional application No. 63/582,685, filed on Sep. 14, 2023.

(51) Int. Cl. A61M 16/06; A61M 16/20

(52) U.S. Cl. CPC A61M 16/0672; A61M 16/202; A61M 2205/8206

(57) ABSTRACT

An electronic nasal cannula flame detector and oxygen shut-off device may include a nasal cannula with a pair of outlets, each outlet being configured for insertion into a nostril of the user. A flow tube has an inlet in fluid communication with the oxygen source and one outlet in fluid communication with the inlet of the cannula. An electronic shut-off valve is disposed in the flow tube and movable from a first position allowing oxygen flow to a second position preventing oxygen flow. A wire conductor provides power from a battery to maintain the electronic shut-off valve in the open position. A portion of the wire conductor extends across at least one outlet of the cannula and is configured to burn in the presence of fire.

### CROSS-REFERENCE TO RELATED APPLICATION

This application claims priority to and the benefit of the filing date of U.S. Provisional Patent Application No. 63/582,685, filed Sep. 14, 2023, the entirety of which is hereby incorporated by reference herein.

### FIELD

This application is generally related to medical devices and procedures and, more particularly, to oxygen therapy devices and methods of use thereof that facilitate the detection of flames within the oxygen systems and secure the oxygen source.

### BACKGROUND

At-home supplemental oxygen therapy is a common treatment for those who suffer from illnesses that affect a person's lung function, such as Chronic Obstructive Pulmonary Disease (COPD). Such illnesses are frequently related to long-term cigarette smoking. During oxygen therapy, oxygen is delivered through a nasal cannula 4 or mask worn by the patient. The cannula 4, or mask, is attached to a source of oxygen 2, such as a pressurized oxygen cylinder or an oxygen concentrator which pulls oxygen from the surrounding air for delivery to the patient. An oxygen-enriched environment lowers the combustion temperatures of materials and allows them to burn hotter and more vigorously. Therefore, smoking poses a risk to those that are actively undergoing oxygen therapy. Typically, before at-home oxygen therapy is commenced, the prospective patient is trained on the dangers of smoking while undergoing treatment, and those patients that are prior smokers often undergo cessation training.

Even with education and training, accidents due to smoking while on oxygen therapy are fairly common occurrences. Such accidents may lead to loss of property due to home fires, severe flash burns to the patient, and even death of the patient. Smoking can be an addictive behavior that is extremely hard for many patients to cease. This fact can be compounded by the advanced age of many patients, as well as accompanying issues such as Alzheimer's, dementia, etc. Devices exist, such as fire stop valves 8 as shown in FIG. 3, that are positioned in the oxygen delivery tube 6 between the oxygen source 2 and the patient. A typical fire stop valve 8 includes a thermal fuse that melts when exposed to fire, causing the valve to secure the flow of oxygen to the patient. One issue with fire stop valves 8 is the fact that the fire must travel up the oxygen delivery tube 6 before reaching the thermal fuse. As such, the fire is supplied by the flow of oxygen until the flame travels the length of tubing and reaches the fire stop valve 8. As well, the flow of oxygen continues for the amount of time required for the fire to travel that length of tubing. During this time, severe burns can be sustained by the 1,000° F. burning Polyvinyl Chloride (PVC) tubing. Due to the location of the tubing, inhalation burns are possible and may be exacerbated by the release of toxic hydrogen chloride gas from the burning PVC tubing.

From the foregoing, one recognizes the need for devices and methods for securing the flow of oxygen to a patient when flames are detected in close proximity to the oxygen delivery system.

### SUMMARY

Embodiments of the disclosed electronic nasal cannula flame detector and oxygen shut-off device may include a user interface device with a body portion having at least one inlet and at least one outlet, wherein each outlet of the at least one outlet is configured for insertion into a corresponding nostril of the user. A flow tube has an inlet and at least one outlet, the inlet of the flow tube being in fluid communication with the oxygen source and each outlet of the at least one outlet of the flow tube being in fluid communication with a corresponding inlet of the user interface device. An electronic shut-off valve is disposed in the flow tube and movable from a first position allowing oxygen flow through the flow tube to a second position preventing oxygen flow through the flow tube. A wire conductor provides power from a battery to the electronic shut-off valve so that when power is provided to the shut-off valve, the shut-off valve is in the first position, and when no power is provided to the shut-off valve, the shut-off valve is in the second position. A first portion of the wire conductor extends across (e.g., spans) the at least one outlet of the user interface device and is configured to burn in the presence of fire.

Also disclosed herein, in some embodiments, is a flame detector and oxygen shut-off valve having a source of oxygen and a user interface device with a body portion including at least one inlet and at least one outlet, wherein each outlet of the at least one outlet is configured to allow the flow of oxygen to the user. A flow tube has an inlet and at least one outlet, the inlet of the flow tube being in fluid communication with the oxygen source and each outlet of the flow tube being in fluid communication with a corresponding inlet of the user interface. An electronic shut-off valve is disposed in the flow tube and movable from a first position that allows oxygen flow through the flow tube to a second position that prevents oxygen flow through the flow tube. A wire conductor provides power from a power source to an electronic shut-off valve so that when power is provided to the shut-off valve, the shut-off valve is in the first position, and when no power is provided to the shut-off valve, the shut-off valve is in the second position. A first portion of the wire conductor extends across (e.g., spans) the at least one outlet of the user interface device and is configured to burn in the presence of fire.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a schematic representation of an oxygen delivery system including an electronic nasal cannula flame detector and oxygen shut-off valve in accordance with an embodiment of the present invention.

FIG. 2 is a detailed view of the nasal cannula of the flame detector and oxygen shut-off valve shown in FIG. 1.

FIG. 3 is schematic representation of an oxygen delivery system including a prior art fire break valve.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents unless the context clearly dictates otherwise. For example, unless the context dictates otherwise, use of the term “a loop” can represent disclosure of embodiments in which only one of such loops is provided, as well as embodiments in which a plurality of such loops are provided.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, in alternative aspects, can also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

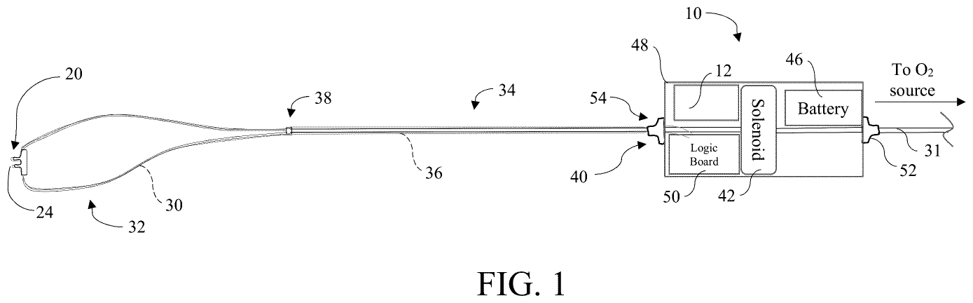

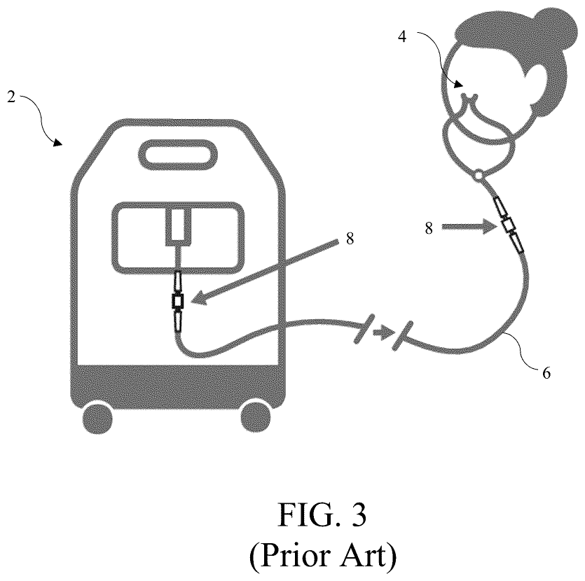

Referring now to FIG. 1, an oxygen delivery system (e.g., an at-home oxygen delivery system) including an electronic nasal cannula flame detector and oxygen shutoff device 10 in accordance with an embodiment of the present disclosure is shown. The oxygen delivery system includes an oxygen source such as a pressurized oxygen cylinder or an oxygen concentrator 2 (FIG. 3) which pulls oxygen from the surrounding air for delivery to the patient. Oxygen is delivered from the oxygen source to a user interface device such as a mask or nasal cannula 20. As is known, a mask is fitted over the mouth and nose of a user, whereas a nasal cannula 20 includes a pair of prongs 24 that is fitted within the nostrils of the user. The presently disclosed embodiment of the electronic oxygen shut off device 10 includes a nasal cannula device 20, although alternate embodiments may utilize a mask. Lengths of flow tubing are utilized to deliver the oxygen from the oxygen source to the nasal cannula device 20. Specifically, in the present embodiment, a first length of main flow tubing 31 extends from the oxygen source to the electronic oxygen shutoff device 10, and a second length of main flow tubing 34 extends from the electronic oxygen shutoff device 10 to a flow tubing loop 32 that is in fluid communication with the nasal cannula device 20.

Still referring to FIG. 1, the electronic oxygen shutoff device 10 is positioned in line with the first and second lengths of main flow tubing 31 and 34 that extend between the oxygen source and the flow tubing loop 32, on which the nasal cannula device 20 is disposed. The electronic oxygen shutoff device 10 includes a housing 48 with an inlet 52 that is in fluid communication with the first length of main flow tubing 31 from the oxygen source. The housing 48 defines an interior compartment that houses a shutoff valve 42 (e.g., a solenoid-operated shutoff valve), a battery 46, a logic board 50, and a battery level indicator light 12. The housing 48 of the electronic oxygen shutoff device 10 also includes an outlet 54 that is in fluid communication with the second length of main flow tubing 34 that is in turn in fluid communication with the flow tubing loop 32 at a juncture 38.

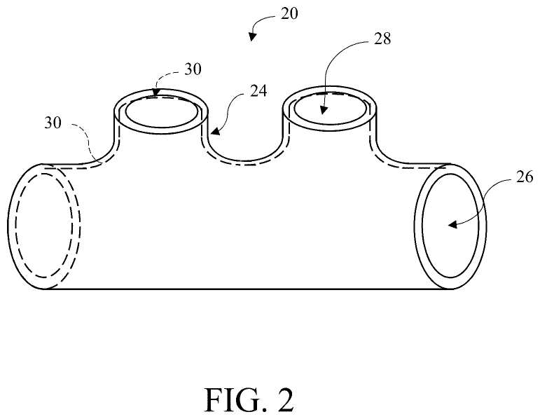

As best seen in FIG. 2, the nasal cannula device 20 includes a body portion 22 having a through bore 26 passing therethrough and a pair of nasal prongs 24 that is configured for insertion into the nostrils of a user. Each nasal prong 24 includes an outlet opening 28 and is in fluid communication with the through bore 26 of the body portion 22. The two openings defined by the through bore 26 are each in fluid communication with a portion of the flow tubing loop 32 such that oxygen is free to flow from the oxygen source into both sides of the nasal cannula device 20.

As shown, the shut-off valve 42 is in line with a portion of the main flow tubing 31 and is moveable from a first position in which the valve is open, thereby allowing oxygen flow through the main flow tubing, to a second position in which the shut-off valve is closed, thereby preventing oxygen flow through the main flow tubing. When the shut-off valve 42 is provided as a solenoid operated shut-off valve, the shut-off valve is preferably operated in a fail-safe condition, meaning that the shut-off valve 42 remains in the open position only as long as the solenoid receives power from the battery 46. The circuit by which the battery 46 provides power to the solenoid operated shut-off valve 42 includes a wire loop (also referred to herein as a “wire conductor”) formed by a first portion 36 that is included in the wall of the main flow tubing 34 and a second portion 30 that is included in the flow tubing loop 32. Specifically, and referring additionally to FIG. 2, the portion of the circuit 30 that is disposed in the flow tubing loop 32 is formed by a light gauge wire, such as, for example and without limitation, a 40 AWG wire, that is disposed within the wall of the flow tubing that forms the tubing loop 32 and extends along the body portion 22 of the nasal cannula device 20. In some aspects, copper wire can be used for the first and the second portions of the circuit 36 and 30, although wire formed of other materials may be used. Although 40 AWG wire is disclosed as a specific example herein, it is contemplated that other gauges can be used, provided the wire can burn rapidly (e.g., in less than 0.25 seconds, or less than 0.04 seconds) in the presence of flames as further disclosed herein. Optionally, it is contemplated that the light gauge wire can have a size ranging from 28 AWG to 56 AWG, or from 36 AWG to 42 AWG, or equal to or smaller than 40 AWG.

As shown in FIG. 2, the light gauge (e.g., 40 AWG) wire extends across the outlet openings 28 defined at the tips of the nasal prongs 24. This placement is desirable to protect a user from potential burns should the oxygen being delivered to the user be ignited. Specifically, were a patient to smoke while oxygen is flowing through the nasal cannula device 20 and the oxygen become ignited, the outlet openings 28 of the nasal prongs 24 are the points at which ignition would occur. The light gauge of the first portion of the circuit wire 30 is selected so that it burns rapidly in the presence of flames. For example, the light gauge (e.g., 40 AWG) wire can be configured to burn through in less than 0.25 seconds. Lighter gauge wire maybe used to obtain quicker burning, and thereby breaking of the circuit, yet fragility of the wires as related to normal usage of the device should be taken into account as well. The first portion 36 of the circuit that extends along the length of the main flow tubing 34 can be selected to be of a larger gauge than is the second wire portion 30 that is designed to break in the presence of flames. The heavier gauge wire (e.g., 22-24 AWG wire) is selected to decrease electrical resistance and increase durability because only one break is required in the circuit to shut off power to the solenoid operated shut-off valve 42. Preferably, a battery level indicator light 12 is provided on the housing 48 of the electronic oxygen shut-off device 10 to indicate the adequate battery power is present to maintain the solenoid operated shut-off valve 42 in the open and operable positions.

Note, although the discussed embodiments include circuits that are broken in the presence of flames to secure oxygen flow, alternate embodiments may forgo these circuits and utilize sensitive smoke detectors to provide signals to the logic board 50 of the electronic oxygen shut-off device 10 to close the shut-off valve 42. Additionally, in some alternate embodiments, both fire-sensitive circuits 31, 36 and smoke detectors may be utilized for redundancy.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system for delivery of oxygen from an oxygen source to nostrils of a user, comprising: a user interface device with a body portion including at least one inlet and at least one outlet, each outlet of the at least one outlet being configured for insertion into a corresponding nostril of the user; a flow tube having an inlet and at least one outlet, the inlet of the flow tube being in fluid communication with the oxygen source and each outlet of the at least one outlet of the flow tube being in fluid communication with a corresponding inlet of the at least one inlet of the user interface device; an electronic shut-off valve disposed in the flow tube and movable from a first position allowing oxygen flow through the flow tube to a second position preventing oxygen flow through the flow tube; a power source; and a wire conductor configured to provide power to the electronic shut-off valve so that when power is provided to the shut-off valve, the shut-off valve is in the first position, and when no power is provided to the shut-off valve, the shut-off valve is in the second position, wherein a first portion of the wire conductor extends across the at least one outlet of the user interface device.

Aspect 2: The system of aspect 1, wherein the user interface device further comprises a nasal cannula including a pair of nasal prongs, each nasal prong including a respective outlet of the user interface device.

Aspect 3: The system of aspect 2, wherein the flow tube includes a first portion and a second portion, the second portion of the flow tube forming a flow loop with the nasal cannula, and the first portion of the flow tube extending from the second portion of the flow tube to the electronic shut-off valve.

Aspect 4: The system of aspect 2 or aspect 3, wherein the first portion of the wire conductor that extends across the outlets of the nasal prongs of the user interface device has a first gauge that is configured to burn through in the presence of fire, thereby stopping delivery of power to the electronic shut-off valve.

Aspect 5: The system of aspect 4, wherein the first gauge of the first portion of the wire conductor is equal to or less than 40 AWG.

Aspect 6: The system of aspect 4 or aspect 5, wherein the wire conductor further comprises a second portion having a second gauge, the second gauge being greater than the first gauge.

Aspect 7: The system of any one of aspects 4-6, wherein the first portion of the wire conductor that extends across the outlets of the nasal prongs is configured to burn through in less than 0.25 seconds.

Aspect 8: The system of any one of aspects 2-7, further comprising a housing, wherein the power source comprises a battery, wherein the electronic shut-off valve and the battery are disposed within the housing.

Aspect 9: The system of any one of aspects 2-8, wherein the wire conductor is embedded or co-extruded within a side wall of the flow tube.

Aspect 10: A system for delivery of a flow of oxygen to a user, comprising: a source of oxygen; a user interface device with a body portion including at least one inlet and at least one outlet, each outlet of the at least one outlet being configured for allowing the flow of oxygen to the user; a flow tube having an inlet and at least one outlet, the inlet of the flow tube being in fluid communication with the oxygen source and each outlet of the at least one outlet of the flow tube being in fluid communication with a corresponding inlet of the at least on inlet of the user interface; an electronic shut-off valve disposed in the flow tube and movable from a first position that allows oxygen flow through the flow tube to a second position that prevents oxygen flow through the flow tube; a power source; and a wire conductor configured to provide power to the electronic shut-off valve so that when power is provided to the shut-off valve, the shut-off valve is in the first position, and when no power is provided to the shut-off valve, the shut-off valve is in the second position, wherein a first portion of the wire conductor extends across the at least one outlet of the user interface device.

Aspect 11: The system of aspect 10, wherein the user interface device is a mask.

Aspect 12: The system of aspect 10, wherein the user interface device comprises a nasal cannula including a pair of nasal prongs, each nasal prong including a respective outlet of the at least one outlet.

Aspect 13: The system of aspect 12, wherein the flow tube includes a first portion and a second portion, the second portion of the flow tube forming a flow loop with the nasal cannula, and the first portion of the flow tube extending from the second portion of the flow tube to the electronic shut-off valve.

Aspect 14: The system of aspect 12 or aspect 13, wherein the first portion of the wire conductor that extends across the at least one outlet of the nasal prongs of the user interface device has a first gauge that is configured to burn through in the presence of fire, thereby stopping delivery of power to the electronic shut-off valve.

Aspect 15: The system of any one of aspects 10-14, wherein the wire conductor further comprises a second portion having a second gauge, the second gauge being greater than the first gauge.

Aspect 16: The system of aspect 15, wherein the first gauge of the first portion of the wire conductor is equal to or less than 40 AWG.

Aspect 17: The system of aspect 15, wherein the first portion of the wire conductor that extends across the outlets of the nasal prongs is configured to burn through in less than 0.25 seconds.

Aspect 18: The system of any one of aspects 10-17, further comprising a housing, wherein the power source comprises a battery, wherein the electronic shut-off valve and the battery are disposed within the housing.

Aspect 19: The system of any one of aspects 10-18, wherein the oxygen source comprises at least one of an oxygen bottle or an oxygen concentrator.

Aspect 20: The system of any one of aspects 10-19, further comprising an oxygen regulator disposed between the oxygen source and the flow tube, wherein the electronic shut-off valve is disposed within the oxygen regulator.

Although the foregoing invention has been described in some detail by way of illustration and example for purposes of clarity and understanding, certain changes and modifications may be practiced within the scope of the suspended claims.

What is claimed is:

1. A system for delivery of oxygen from an oxygen source to nostrils of a user, comprising:
a user interface device with a body portion including at least one inlet and at least one outlet, each outlet of the at least one outlet being configured for insertion into a corresponding nostril of the user;
a flow tube having an inlet and at least one outlet, the inlet of the flow tube being in fluid communication with the oxygen source and each outlet of the at least one outlet of the flow tube being in fluid communication with a corresponding inlet of the at least one inlet of the user interface device;
an electronic shut-off valve disposed in the flow tube and movable from a first position allowing oxygen flow through the flow tube to a second position preventing oxygen flow through the flow tube;
a power source; and
a wire conductor configured to provide power to the electronic shut-off valve so that when power is provided to the shut-off valve, the shut-off valve is in the first position, and when no power is provided to the shut-off valve, the shut-off valve is in the second position,
wherein a first portion of the wire conductor extends across the at least one outlet of the user interface device.

2. The system of claim 1, wherein the user interface device further comprises a nasal cannula including a pair of nasal prongs, each nasal prong including a respective outlet of the user interface device.

3. The system of claim 2, wherein the flow tube includes a first portion and a second portion, the second portion of the flow tube forming a flow loop with the nasal cannula, and the first portion of the flow tube extending from the second portion of the flow tube to the electronic shut-off valve.

4. The system of claim 2, wherein the first portion of the wire conductor that extends across the outlets of the nasal prongs of the user interface device has a first gauge that is configured to burn through in the presence of fire, thereby stopping delivery of power to the electronic shut-off valve.

5. The system of claim 4, wherein the first gauge of the first portion of the wire conductor is equal to or less than 40 AWG.

6. The system of claim 4, wherein the wire conductor further comprises a second portion having a second gauge, the second gauge being greater than the first gauge.

7. The system of claim 4, wherein the first portion of the wire conductor that extends across the outlets of the nasal prongs is configured to burn through in less than 0.25 seconds.

8. The system of claim 2, further comprising a housing, wherein the power source comprises a battery, wherein the electronic shut-off valve and the battery are disposed within the housing.

9. The system of claim 2, wherein the wire conductor is embedded or co-extruded within a side wall of the flow tube.

10. A system for delivery of a flow of oxygen to a user, comprising:
a source of oxygen;
a user interface device with a body portion including at least one inlet and at least one outlet, each outlet of the at least one outlet being configured for allowing the flow of oxygen to the user;
a flow tube having an inlet and at least one outlet, the inlet of the flow tube being in fluid communication with the oxygen source and each outlet of the at least one outlet of the flow tube being in fluid communication with a corresponding inlet of the at least on inlet of the user interface;
an electronic shut-off valve disposed in the flow tube and movable from a first position that allows oxygen flow through the flow tube to a second position that prevents oxygen flow through the flow tube;
a power source; and
a wire conductor configured to provide power to the electronic shut-off valve so that when power is provided to the shut-off valve, the shut-off valve is in the first position, and when no power is provided to the shut-off valve, the shut-off valve is in the second position,
wherein a first portion of the wire conductor extends across the at least one outlet of the user interface device.

11. The system of claim 10, wherein the user interface device is a mask.

12. The system of claim 10, wherein the user interface device comprises a nasal cannula including a pair of nasal prongs, each nasal prong including a respective outlet of the at least one outlet.

13. The system of claim 12, wherein the flow tube includes a first portion and a second portion, the second portion of the flow tube forming a flow loop with the nasal cannula, and the first portion of the flow tube extending from the second portion of the flow tube to the electronic shut-off valve.

14. The system of claim 12, wherein the first portion of the wire conductor that extends across the at least one outlet of the nasal prongs of the user interface device has a first gauge that is configured to burn through in the presence of fire, thereby stopping delivery of power to the electronic shut-off valve.

15. The system of claim 10, wherein the wire conductor further comprises a second portion having a second gauge, the second gauge being greater than the first gauge.

16. The system of claim 15, wherein the first gauge of the first portion of the wire conductor is equal to or less than 40 AWG.

17. The system of claim 15, wherein the first portion of the wire conductor that extends across the outlets of the nasal prongs is configured to burn through in less than 0.25 seconds.

18. The system of claim 10, further comprising a housing, wherein the power source comprises a battery, wherein the electronic shut-off valve and the battery are disposed within the housing.

19. The system of claim 10, wherein the oxygen source comprises at least one of an oxygen bottle or an oxygen concentrator.

20. The system of claim 10, further comprising an oxygen regulator disposed between the oxygen source and the flow tube, wherein the electronic shut-off valve is disposed within the oxygen regulator.
